(19) United States
(12) Patent Application Publication
(10) Pub. No.: US 2004/0098814 A1
(43) Pub. Date: May 27, 2004

(54) Method and composition for the gradual permanent coloring of hair which employ carbonates

(75) Inventors: Van Au, New City, NY (US); Stephen Alan Madison, New City, NY (US); John Brian Bartolone, Bridgeport, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/306,605

(22) Filed: Nov. 27, 2002

(51) Int. Cl. A61K 7/13

(52) U.S. Cl. 8/405

(57) ABSTRACT

A method for permanently dyeing hair is provided which includes subjecting said hair to a number of treatments having a set time interval between each two consecutive such treatments. In a first step hair is contacted for a period of about 5 seconds to about 5 minutes with a recently prepared mixture of an alkaline composition comprising a dye intermediate in a shampoo or conditioner base, a water soluble carbonate releasing salt, and a water soluble ammonium salt. Alternatively, contact can be with a water soluble ammonium carbonate or carbamate salt and a peroxide compound at acidic pH in a conditioner or shampoo base. Thereafter, the hair is rinsed with water. The number of treatments is between about 2 to about 30. Time interval between each two consecutive treatments is between about 8 hours and 30 days.

### BACKGROUND OF THE INVENTION

The present invention relates to a method for the permanent coloring of hair with longer lasting conditioning and with minimized hair damage.

Most hair coloring products fall under three major groupings:

1. Temporary hair color

2. Semi permanent hair color

3. Permanent hair color

Temporary color is a leave on product that causes minimal damage to the hair. However, temporary color causes stains, and leaches out under rain or with perspiration. Temporary color washes out with the next shampoo. Temporary color also does not give any control to the consumer over the amount of color deposited or the permanency of the color supplied. Temporary color does not result in a wide variety of colors and it has only a limited appeal.

Semi-permanent hair color comes as a rinse, and it causes minimal damage to the hair. However, semi-permanent hair color washes out to some degree with each shampoo and washes out completely within about 4 to 6 shampoos. Semi permanent hair color does not give the consumer any control regarding the amount of color deposited or the permanency of the color. Semi-permanent hair color has limited popularity with consumers.

Permanent hair color generally comes in two parts: a dye solution and a developer solution. Because of the damaging nature of current permanent dye treatments, most home coloring products come with a post treatment conditioner. In a permanent hair coloring treatment, the dye solution and the developer solution are mixed and then applied to the hair, which is then left for about 25 to about 35 minutes. The hair is then rinsed with water, treated with a post treatment conditioner, and then rinsed again with water.

The application of the dye solution and the developer solution affords permanent hair coloring. However, this method does not provide any conditioning benefit. The conditioning benefit comes through application of the post treatment conditioner, and it is only temporary. The conditioning benefit is lost with the next shampoo. Moreover, with permanent hair coloring treatments, shampooing the hair is usually not recommended after said treatments. Thus, hair is left feeling dirty, and can stain towels and pillows.

Coloring products need to be applied every four to six weeks since hair grows out of the scalp at the rate of approximately one half inch per month. Each coloring application causes damage to the hair, and that damage is cumulative. Hair touch ups after the initial treatment would also damage hair more.

It would be desirable to develop a method for permanently and effectively coloring hair that conditions hair, gives hair a soft clean feel, and minimizes the damage caused to hair by the coloring process. The present invention provides such a method.

Current hair coloring products cannot be used safely in the shower. It is an object of this invention to develop a method for permanently coloring hair, which can be carried out safely in the shower. It is also an object of the invention to provide a method for permanently coloring hair wherein the user has control of the amount of durable color deposited without hair damage. It is also an object of the invention to provide a method for permanently coloring hair wherein the user has control over the amount of lifting/lightening without causing hair damage. It is also an object of the invention to provide a method for permanently coloring hair wherein the user can employ the product as her daily hair care product to avoid new outgrowth of uncolored hair. It is also an object of the invention to provide a method for permanently coloring hair wherein said method involves less mess and difficulty than conventional permanent hair coloring methods. It is also an object of the invention to provide a method for permanently coloring hair wherein said method brings about gradual color changes with each application. Since gradual color changes are to occur, such a method would be virtually mistake free because the consumer could stop or alter the coloring method if she did not like the course the hair coloring was taking. It is also an object of the invention to provide a method for permanently coloring hair wherein the amount of hair coloring composition employed can be varied from application to application in order to adjust the hair coloring results.

These and other aspects of this invention will become evident by a detailed description of the invention given below.

Patents related to the field of this invention are as follows:

U.S. Pat. No. 5,131,912 discloses durable 2-part hair dyeing agents composed of a first agent comprising as essential components at least one compound that forms HCO₃— by dissociation in water, an alkali generating substantially no irritating odor and a dye for hair and having a pH of 8.2 to 9.0. and a second agent comprising as essential components hydrogen peroxide and a buffer solution and having a pH of 2.0 to 4.0, the weight ratio of the first agent and the second agent to be mixed being such that the pH of the mixture of the two is in a range of from 6.5 to 7.9. These 2-part hair dyeing agents require only a short dyeing time, create little damage to hair and no irritating or disagreeable odor and have high dyeing effect.

U.S. Pat. No. 5,525,123, discloses a hair dyeing composition based on oxidation dyestuff precursors which dyes and brightens the hair containing, besides at least one developing and at least one coupling agent, at least one metal salt and at least one ammonium compound selected from the group ammonium chloride, ammonium sulfate, ammonium carbonate, ammonium bicarbonate, and ammonium carbamate, having a pH-value between 8 and 11, preferably from 9 to 10, after admixture with an oxidizing agent in the ready-to-use preparation.

Ser. No. 10/090,715 filed Mar. 5, 2002, a method for permanently dyeing hair which comprises subjecting said hair to a number of treatments, having a set time interval between each two consecutive such treatments, wherein each treatment comprises steps a.) and b.) below:

a.) contacting said hair, for a period of about 5 seconds to about 5 minutes with a recently made mixture of:

A) an alkaline composition comprising a dye intermediate in a shampoo base or in a conditioner base; and

B) an acidic composition comprising an oxidating compound in a shampoo base or in a conditioner base;

b.) rinsing said mixture from said hair with water;

with the proviso that when a conditioner base is present in A above, an independently selected conditioner base is also present in B above; and when a shampoo base is present in A above, an independently selected shampoo base is also present in B above;

and wherein said number of treatments is between about 2 to about 30; and wherein said set time interval between each two consecutive treatments is between about 8 hours and 30 days, is described.

U.S. Pat. No. 4,104,021 discloses a process in which human hair is dyed in successive treatments at selected intervals with oxidation colors (aromatic primary amines and amino phenols) admixed in each treatment with an oxidizing agent (H₂O₂ or a derivative thereof)—the quantity of oxidation colorant applied in each treatment being substantially the same and the quantity of oxidizing agent being increased from the first to the last treatment to effect a gradual increase in depth of shade—the mixture being allowed to remain on the hair for substantially the same time in each treatment, followed by removal by rinsing.

U.S. Pat. No. 4,529,404 discloses an autoxidizable hair dye preparation capable of coloring or darkening hair when applied thereto and exposed to the atmosphere comprising a mixture of (I) at least one p-phenylene diamine compound, or an acid addition salt thereof, and (II) at least one 1,2,4-benzenetriol compound, each compound optionally containing nuclearly substituted C₁₋₄ alkyl, alkoxy, hydroxyalkyl or halogen. The preparation is preferably applied and exposed to the atmosphere repeatedly until the desired degree of darkening or color build-up is attained.

The preparations of this invention may also contain known additives or assistants such as hair grooming agents, for example quaternized vinyl pyrrolidone copolymers, carboxyvinyl polymers and the like, plasticizers, conditioners, thickeners, slip and wetting agents such as polyoxyethylenated fatty (e.g. lauryl) alcohols, stearyldimethylammonium chloride, silicone copolymer, foam boosters, preservatives, perfumes and the like.

U.S. Pat. No. 5,968,486 describes a shampoo composition for lightening and highlighting hair which comprises

(i) a peroxygen compound; and

(ii) an anionic sulfonate;

said composition having a pH less than 5. There is also described an invention directed to a method for lightening and highlighting hair which comprises shampooing the hair with a lightening and highlighting effective amount of a composition of the invention.

Co-pending and commonly assigned U.S. patent application Ser. No. 09/558,235, filed Apr. 24, 2000 discloses a hair conditioning composition for conditioning, lightening, and hilighting hair, which comprises

i) peroxygen compound, and

ii) a conditioning agent, said composition having a pH of 5 or less.

### SUMMARY OF THE INVENTION

The invention relates to a method for achieving permanent desired hair color change through the use of daily hair care compositions. The daily hair care compositions comprise a mixture of two compositions, part A and part B as described just below:

Part A:

I Dye intermediates in a conditioner or shampoo base at alkaline pH, and mixture of:

I an alkaline composition comprising a dye intermediate in a shampoo base or in a conditioner base and

II

i) a water soluble carbonate releasing salt at about 0.1% to about 15%, more preferably about 1% to about 10%, and more preferably about 4% to about 10%, and most preferably about 8%; and

i) a water soluble ammonium salt at about 0.1% to about 15%, more preferably about 1% to about 10%, and more preferably about 4% to about 10%, and most preferably about 8%; or

II

a water soluble ammonium carbonate or carbamate salt at about 0.1% to about 15%, more preferably about 1% to about 10%, and more preferably about 4% to about 10%, and most preferably about 8%; and

III

Part B: A peroxide compound such as hydrogen peroxide in a conditioner or shampoo base at acidic pH;

The present invention also relates to a method for coloring hair which comprises contacting said hair with a recently made mixture of part A and part B of the invention on a regular or daily basis.

The present invention relates to specific compositions that are a mixture of part A and part B as described just below.

### DETAILED DESCRIPTION OF THE INVENTION

As used here % means weight % of the total composition unless otherwise specified. When the words “about 0.1 to about 15% an carbonate salt, or about 0.1 to about 15%, of a metal carbonate salt and about 0.1 to about 15%, ammonium salt or whenever % of carbonates or ammonium salts are specified this means weight % relative to the total of part A of the composition.” As used herein the term “recently” means within a very short interval of time such as within a few seconds or minutes, such as within 0.01 seconds to 120 seconds, or within 0.1 seconds to 60 seconds, or within 0.5 second to within 30 second or within 2 seconds to within 20 seconds. Composition of the invention may be made by methods which are known in the art or which are analogous to those which are known in the art. Compositions of the invention may be made from ingredients which are known in the art or which are analogous to those which are known in the art.

Part A compositions of the invention may comprise the following materials:

Part A: Dye intermediates in a conditioner or shampoo base at alkaline pH;

In the case of compositions in a conditioner base, Part A may comprise from

a) about 0.1 to about 99.9% of a conditioning base, wherein the conditioning agent within said conditioning base comprises from about 0.5 to about 10% of the total composition % of a conditioner;

b) about 0.1 to about 5% of oxidation dyestuffs;

c) about 0.1 to about 5% of a coupling compound; and

I an alkaline composition comprising a dye intermediate in a shampoo base or in a conditioner base and

II

j) a water soluble carbonate releasing salt at about 0.1% to about 15%, more preferably about 1% to about 10%, and more preferably about 4% to about 10%, and most preferably about 8%; and

ii) a water soluble ammonium salt at about 0.1% to about 15%, more preferably about 1% to about 10%, and more preferably about 4% to about 10%, and most preferably about 8%; or

II

a water soluble ammonium carbonate or carbamate salt at about 0.1% to about 15%, more preferably about 1% to about 10%, and more preferably about 4% to about 10%, and most preferably about 8%; and

Part B: a peroxide such as hydrogen peroxide in a conditioner or shampoo base at acidic pH.

The invention also relates to methods for coloring hair which comprise contacting the hair with a recently made mixture of part A and part B of the invention.

More preferably, in the case of compositions in a conditioner base, part A may comprise from

a) about 1 to about 5% of a conditioning agent;

b) about 0.1 to about 0.5% of dye;

c) about 0.1 to about 1% coupling compound; and

mixture of:

I an alkaline composition comprising a dye intermediate in a shampoo base or in a conditioner base and

II

k) a water soluble carbonate releasing salt at about 0.1% to about 15%, more preferably about 1% to about 10%, and more preferably about 4% to about 10%, and most preferably about 8%; and

ii) a water soluble ammonium salt at about 0.1% to about 15%, more preferably about 1% to about 10%, and more preferably about 4% to about 10%, and most preferably about 8%; or

II

a water soluble ammonium carbonate or carbamate salt at about 0.1% to about 15%, more preferably about 1% to about 10%, and more preferably about 4% to about 10%, and most preferably about 8%; and

More preferably, in the case of compositions in a conditioner base, part A can comprise from

a) about 1 to about 5% of a conditioning agent;

b) about 0.1 to about 0.5% of dye;

II

l) a water soluble carbonate releasing salt at about 0.1% to about 15%, more preferably about 1% to about 10%, and more preferably about 4% to about 10%, and most preferably about 8%; and

iii) a water soluble ammonium salt at about 0.1% to about 15%, more preferably about 1% to about 10%, and more preferably about 4% to about 10%, and most preferably about 8%; or

II

a water soluble ammonium carbonate or carbamate salt at about 0.1% to about 15%, more preferably about 1% to about 10%, and more preferably about 4% to about 10%, and most preferably about 8%;

More preferably for compositions in a conditioner base, Part B comprises from:

a) about 1 to about 5% of a conditioning agent; and

b) about 1 to about 5% of an oxidizing compound.

For compositions in a shampoo base part A comprises:

a) about 0.1 to about 99.9% of a shampoo base, wherein the detergent foaming agent within said shampoo base comprises from about 0.5 to about 50% of the total composition % of a shampoo agent;

b) about 0.1 to about 5% of oxidation dyestuffs;

c) about 0.1 to about 5% of a coupling compound.

mixture of:

I an alkaline composition comprising a dye intermediate in a shampoo base or in a conditioner base and

II

m) a water soluble carbonate releasing salt at about 0.1% to about 15%, more preferably about 1% to about 10%, and more preferably about 4% to about 10%, and most preferably about 8%; and

iv) a water soluble ammonium salt at about 0.1% to about 15%, more preferably about 1% to about 10%, and more preferably about 4% to about 10%, and most preferably about 8%; or

II

a water soluble ammonium carbonate or carbamate salt at about 0.1% to about 15%, more preferably about 1% to about 10%, and more preferably about 4% to about 10%, and most preferably about 8%;

More preferably, in the case of compositions in a shampoo base, part A may comprise from

a) about 2 to about 15% of a detergent foaming agent;

b) about 0.1 to about 0.5% of dye;

c) about 0.1 to about 1% coupling compound.

II

n) a water soluble carbonate releasing salt at about 0.1% to about 15%, more preferably about 1% to about 10%, and more preferably about 4% to about 10%, and most preferably about 8%; and

v) a water soluble ammonium salt at about 0.1% to about 15%, more preferably about 1% to about 10%, and more preferably about 4% to about 10%, and most preferably about 8%; or

II

a water soluble ammonium carbonate or carbamate salt at about 0.1% to about 15%, more preferably about 1% to about 10%, and more preferably about 4% to about 10%, and most preferably about 8%;

d) a volatile silicone;

Part B comprises:

a) about 1 to about 5% of a conditioning base;

b) about 1 to about 5% of an oxidative or a peroxide compound.

A conditioning composition of the invention is a composition as described just above, which further comprises in part A, part B, or part A and part B, a thickener which is a high molecular weight fatty alcohol wherein said high molecular weight fatty alcohol is selected from the group consisting of cetyl alcohol and stearyl alcohol.

A shampoo composition for permanently dying hair comprises a mixture of Part A and part B wherein:

Part A comprises:

a) about 0.1% to about 99.9% of a shampoo base; and

b) about 0.1% to about 1% of a dye; and

II

o) a water soluble carbonate releasing salt at about 0.1% to about 15%, more preferably about 1% to about 10%, and more preferably about 4% to about 10%, and most preferably about 8%; and

vi) a water soluble ammonium salt at about 0.1% to about 15%, more preferably about 1% to about 10%, and more preferably about 4% to about 10%, and most preferably about 8%; or

II

a water soluble ammonium carbonate or carbamate salt at about 0.1% to about 15%, more preferably about 1% to about 10%, and more preferably about 4% to about 10%, and most preferably about 8%.

Part B comprises:

a) about 1 to about 5% of a shampoo base;

b) about 1 to about 5% of an oxidative or a peroxide compound.

A shampoo composition of the invention is as described just above wherein said shampoo base in part A comprises about 5 to about 50%, based on the total part A composition, of an anionic surfactant, an amphoteric surfactant, or a mixture of an anionic surfactant and an amphoteric surfactant.

What follows is a description of the ingredients that can be included in the compositions of the present invention.

Hair Dyes and Hair Coloring Agents

The part A compositions of the present invention include as an essential feature one or more oxidative hair coloring agents. These hair coloring agents are present in compositions of the present invention which have a conditioner base, and compositions of the present invention which have a shampoo base. Such oxidative hair-coloring agents are used in combination with the oxidizing systems of the present invention to formulate permanent hair dye compositions.

Permanent hair dye compositions as defined herein are compositions, which once applied to the hair, are substantially resistant to washout.

Oxidative Dyes

The dye forming intermediates used in oxidative dyes are essentially aromatic diamines, aminophenols and their derivatives. These dye forming intermediates can be classified as; primary and secondary intermediates, couplers and modifiers, and nitro dyes. Primary intermediates are chemical compounds, which by themselves will form a dye upon oxidation. The secondary intermediates, also known as color modifiers or couplers and are used with other intermediates for specific color effects or to stabilize the color. Nitro dyes are unique in that they are direct dyes, which do not require oxidation to dye the hair.

The oxidation dye intermediates, which are suitable for, use in the compositions and processes herein include aromatic diamines, polyhydric phenols, aminophenols and derivatives of these aromatic compounds (e.g., N-substituted derivatives of the amines, and ethers of the phenols). Primary oxidation dye intermediates are generally colorless molecules prior to oxidation. The oxidation dye color is generated when the primary intermediate is ‘activated’ and subsequently enjoined with a secondary intermediate (coupling agent), which is also generally colorless, to form a colored, conjugated molecule. In general terms, oxidation hair dye precursors or intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear colored. For example, oxidation dye precursors capable of forming colored polymers include materials such as aniline, which has a single functional group and which, on oxidation, forms a series of conjugated imines and quinoid dimers, trimers, etc. ranging in color from green to black. Compounds such as p-phenylenediamine, which has two functional groups, are capable of oxidative polymerization to yield higher molecular weight colored materials having extended conjugated electron systems. Color modifiers (couplers), such as those detailed hereinafter, are preferably used in conjunction with the oxidation dye precursors herein and are thought to interpose themselves in the colored polymers during their formation and to cause shifts in the electronic spectra thereof, thereby resulting in slight color changes. A representative list of oxidation dye precursors suitable for use herein is found in Sagarin, “Cosmetic Science and Technology”, “Interscience, Special Edition, Volume 2, pages 308 to 310.

It is to be understood that the oxidizing aids of the present invention are suitable for use (in combination with a source of peroxide as detailed herein) with all manner of oxidation dye precursors and color modifiers and that the precursors detailed below are only by way of example and are not intended to limit the compositions and processes herein.

The typical aromatic diamines, polyhydric phenols, aminophenols, and derivatives thereof, described above as primary dye precursors can also have additional substituents on the aromatic ring, e.g. halogen, aldehyde, carboxylic additional substituents on the amino nitrogen and on the phenolic oxygen, e.g. substituted and unsubstituted alkyl and aryl groups.

The hair coloring compositions of the present invention may, in addition to the essential oxidative hair-coloring agents, optionally include non-oxidative and other dye materials. Optional non-oxidative and other dyes suitable for use in the hair coloring compositions and processes according to the present invention include semi-permanent, temporary and other dyes. Non-oxidative dyes as defined herein include the so-called ‘direct action dyes’, metallic dyes, metal chelate dyes, fiber reactive dyes and other synthetic and natural Chemical and Physical Behaviour of Human Hair’ 3rd Edn. by Clarence Robbins (pp 250-259); ‘The Chemistry and Manufacture of Cosmetics’. Volume IV. 2nd Edn. Maison G. De dyes. Various types of non-oxidative dyes are

detailed in: ‘Navarre at chapter 45 by G. S. Kass (pp 841-920); ‘cosmetics: Science and Technology’ 2nd Edn, Vol II Balsam Sagarin, Chapter 23 by F. E. Wall (pp 279-343); ‘The Science of Hair Care’ edited by C. Zviak, Chapter 7 (pp 235-261) and ‘Hair Dyes’, J. C. Johnson, Noyes Data Corp., Park Ridge, U.S.A. (1973), (pp 3-91 and 113-139).

Specific hair dyes which may be included in the compositions of the invention include m-aminophenol, p-phenylene diamine, p-toluenediamine; p-phenylenediamine; 2-chloro-p-phenylenediamine; N-phenyl-p-phenylenediamine; N-2-methoxyethyl-p-phenylenediamine; N,N-bis-(hydroxyethyl)-p-phenylenediamine; 2-hydroxymethyl-p-phenylenediamine; 2-hydroxyethyl-p-phenylenediamine; 4,4′-diaminodiphenylamine; 2,6-dimethyl-p-phenylenediamine; 2-isopropyl-p-phenylenediamine; N-(2-hydroxypropyl)-p-phenylenediamine; 2-propyl-p-phenylenediamine; 1,3-N,N-bis-(2-hydroxyethyl)-N,N-bis (4-aminophenyl)-2-propanol; 2-methyl-4-dimethylaminoaniline; p-aminophenol; p-methylaminophenol; 3-methyl-p-aminophenol; 2-hydroxymethyl-p-aminophenol; 2-methyl-p-aminophenol; 2-(2-hydroxyethylaminomethyl)-p-aminophenol; 2-methoxymethyl-p-aminophenol; and 5-aminosalicylic acid; catechol; pyrogallol; o-aminophenol; 2,4-diaminophenol; 2,4,5-trihydroxytoluene; 1,2,4-trihydroxybenzene; 2-ethylamino-p-cresol; 2,3-dihydroxynaphthalene; 5-methyl-o-aminophenol; 6-methyl-o-aminophenol; and 2-amino-5-acetaminophenol; 2-methyl-1-naphthol; 1-acetoxy-2-methylnaphthalene; 1,7-dihydroxynaphthalene; resorcinol; 4-chlororesorcinol; 1-naphthol; 1,5-dihydroxynaphthalene; 2,7-dihydroxynaphthalene; 2-methylresorcinol; 1-hydroxy-6-aminonaphthalene-3-sulfonic acid; thymol (2 isopropyl-5-methylphenol); 1,5-dihydroxy-1,2, 3,4-tetrahydronaphthalene; 2-chlororesorcinol; 2,3-dihydroxy-1,4-naphthoquinone; and 1-naphthol-4-sulfonic acid; m-phenylenediamine; 2-(2,4-diaminophenoxy)ethanol; N,N-bis(hydroxyethyl)-m-phenylenediamine; 2,6-diaminotoluene; N,N-bis(hydroxyethyl)-2,4-diaminophenetole; bis(2,4-diaminophenoxy)-1,3-propane; 1-hydroxyethyl-2,4-diaminobenzene; 2-amino-4 hydroxyethylaminoanisole; aminoethoxy-2,4-diaminobenzene; 2,4-diaminophenoxyacetic acid; 4,6-bis(hydroxyethoxy)-m-phenylenediamine; 2,4-diamino-5-methylphenetole; 2,4-diamino-5-hydroxyethoxytoluene; 2,4-dimethoxy 1,3-diaminobenzene; and 2,6-bis(hydroxyethylamino) toluene; m-aminophenol; 2-hydroxy-4-carbamoylmethylaminotoluene; m-carbamoylmethylaminophenol; 6-hydroxybenzomorpholine; 2-hydroxy-4-aminotoluene; 2-hydroxy-4-hydroxyethylaminotoluene; 4,6-dichloro-m-aminophenol; 2-methyl-m-aminophenol; 2-chloro-6-methyl-m-aminophenol; 2-hydroxyethoxy-5-aminophenol; 2-chloro-5-trifluoroethylaminophenol; 4-chloro-6-methyl-m-aminophenol; N-cyclopentyl-3-aminophenol; N-hydroxyethyl-4-methoxy-2-methyl-m-aminophenol and 5-amino-4-methoxy-2-methylpheno; 2-dimethylamino-5-aminopyridine; 2,4,5,6-tetra-aminopyrimidine; 4,5-diamino-1-methylpyrazole; 1-phenyl-3-methyl-5-pyrazolone; 6-methoxy-8-aminoquinoline; 2,6-dihydroxy-4-methylpyridine; 5-hydroxy-1,4-benzodioxane; 3,4-methylenedioxyphenol; 4-hydroxyethylamino-1,2-methylenedioxybenzene; 2,6-dihydroxy-3,4-dimethylpyridine; 5-chloro-2,3-dihydroxypyridine; 3,5-diamino-2,6-dimethoxypyridine; 2-hydroxyethylamino-6-methoxy-3-aminopyridine; 3,4-methylenedioxyaniline; 2,6-bis-hydroxyethoxy-3,5-diaminopyridine; 4-hydroxyindole; 3-amino-5-hydroxy-2,6-dimethoxypyridine; 5,6-dihydroxyindole; 7-hydroxyindole; 5-hydroxyindole; 2-bromo-4,5-methylenedioxyphenol; 6-hydroxyindole; 3-amino-2-methylamino-6-methoxypyridine; 2-amino-3-hydroxypyridine; 2,6-diaminopyridine; 5-(3,5-diamino-2-pyridyloxy)-1,3-dihydroxypentane; 3-(3,5-diamino-2-pyridyloxy)-2-hydroxypropanol and 4-hydroxy-2,5,6-triaminopyrimidine, or combinations thereof.

Buffering Agents

The coloring compositions of the present invention have a preferred pH in the range of from about 7.5 to about 12, more preferably from about 8 to about 10.

Buffering agents may be present in part A compositions of the present invention which have a conditioner base, and compositions of the present invention which have a shampoo base. Coloring compositions of the present invention may contain one or more hair swelling agents (HSAs) such as urea, to adjust the pH to the desired level. Several different pH modifiers can be used to adjust the pH of the final composition or any constituent part thereof.

Further examples of suitable buffering agents are ammonium hydroxide, urea, ethylamine, dipropylamine, triethylamine and alkanediamines such as 1,3-diaminopropane, anhydrous alkaline alkanolamines such as, mono or diethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-alginine, lysine, oxylysine and histidine and alkanolamines such as dimethylaminoethanol and aminoalkylpropanediol and mixtures thereof.

As herein before described certain alkaline buffering agents such as ammonium hydroxide and monoethylamine (MEA) can also act as hair swelling agents (HSA's) such as urea and the like.

Preferred for use as a buffering agent for the coloring compositions according to the present invention is ammonium hydroxide and/or sodium hydroxide.

In oxidizing and coloring kits comprising a portion of peroxide oxidizing agent, which may be present in either solid or liquid form, such as hydrogen peroxide, a buffering agent solution is required to stabilize hydrogen peroxide. Since hydrogen peroxide is stable in the pH range from 2 to 4, it is necessary to use a buffering agent having a pH within this range. Dilute acids are suitable hydrogen peroxide buffering agents. Phosphoric acid is a preferred agent for buffering hydrogen peroxide solutions.

This pH adjustment can be effected by using well known acidifying agents in the field of treating keratinous fibers, and in particular human hair, such as inorganic and organic acids such as hydrochloric acid, tartaric acid, citric acid, phosphoric acid and carboxylic or sulfonic acids such as ascorbic acid, acetic acid, lactic acid, sulfuric acid, formic acid, ammonium sulfate and sodium dihydrogenphosphate/phosphoric acid, disodium hydrogen phosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate/citric acid, sodium tartarate/tartaric acid, sodium lactate/lactic acid, sodium acetate/acetic acid, disodium hydrogenphosphate/citric acid and sodium chloride/glycine/hydrochloric acid and mixtures thereof.

Solvents

Water is the preferred principal diluent for the compositions according to the present invention. As such, the compositions according to the present invention may include one or more solvents as additional diluent materials. Generally, the solvent is selected to be miscible with water and innocuous to the skin. Solvents suitable for use herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a particularly preferred group includes ethanol, isopropanol, n-propanol, butanol, propylene glycol, ethylene glycol monoethyl ether, and mixtures thereof.

These solvents may be present in compositions of the present invention which have a conditioner base, and in compositions of the present invention which have a shampoo base. These solvents may be present in part A compositions of the invention and part B compositions of the invention.

Surfactant Materials

The compositions of the present invention can additionally contain a surfactant system. Suitable surfactants for inclusion in the compositions of the invention generally have a lipophilic chain length of from about 8 to about 22 carbon atoms. Surfactants which are either amphoteric or anionic or zwitterionic are included in compositions of the present invention which have a shampoo base. In addition, there may be included in shampoo compositions of the present invention, cationic, nonionic or zwitterionic surfactants. Compositions of the invention have a conditioner base can included cationic, nonionic or zwitterionic surfactants. Surfactants may be present in part A and part B compositions of the invention.

(i) Anionic Surfactants

Anionic surfactants suitable for inclusion in the compositions of the invention include alkyl sulfates, ethoxylated alkyl sulfates, alkyl glyceryl ether sulfonates, methyl acyl taurates, fatty acyl glycinates, N-acyl glutamates, acylisethionates, alkyl sulfosuccinates, alkyl ethoxysulfosuccinates, alpha-sulfonated fatty acids, their salts and/or theiresters, alkyl ethoxy carboxylates, alkyl phosphate esters, ethoxylated alkyl phosphate esters, alkyl sulfates, acylsarcosinates and fatty acid/protein condensates, and mixtures thereof. Alkyl and/or acyl chain lengths for these surfactants are C12-C22, preferably C12-C18 more preferably C12-C14.

(ii) Nonionic Surfactants

The compositions of the invention can also comprise a water-soluble nonionic surfactants.

Suitable oil derived nonionic surfactants for use herein include water soluble vegetable and animal-derived emollients such as triglycerides with a polyethyleneglycol chain inserted; ethoxylated mono and di-glycerides, polyethoxylated lanolins and ethoxylated butter derivatives.

Preferred for use herein are polyethyleneglycol based polyethoxylated C₉-C₁₅ fatty alcohol nonionic surfactants containing an average of from about 5 to about 50 ethyleneoxy moieties per mole of surfactant.

Also suitable for use herein are nonionic surfactants derived from composite vegetable fats extracted from the fruit of the Shea Tree (Butyrospermum Karkii Kotschy) and derivatives thereof. Similarly, ethoxylated derivatives of Mango, Cocoa and Illipe butter may be used in compositions according to the invention. Although these are classified as ethoxylated nonionic surfactants it is understood that a certain proportion may remain as non-ethoxylated vegetable oil or fat.

Other suitable oil-derived nonionic surfactants include ethoxylated derivatives of almond oil, peanut oil, rice bran oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, and sunflower seed oil.

(iii) Amphoteric Surfactants

Amphoteric surfactants suitable for use in the compositions of the invention include: alkyl betaines, alkyl sultaines, alkyl amphoacetates, alkyl amphodiacetates, alkyl amphopropionates, and alkyl amphodipropionates.

(iv) Zwitterionic Surfactants

Water-soluble auxiliary zwitterionic surfactants suitable for inclusion in the compositions of the present invention include alkyl betaines

Water-soluble auxiliary sultaine surfactants suitable for inclusion in the compositions of the present invention include alkyl sultaines. Preferred for use herein is coco amido propylhydroxy sultaine.

Water-soluble auxiliary amine oxide surfactants suitable for inclusion in the compositions of the present invention include alkyl amine oxide R.sub.5 R.sub.6 R.sub.7 NO and amido amine oxides number from 1 to 4. Preferred amine oxides include cocoamidopropylamine oxide, lauryl dimethyl amine oxide and myristyl dimethyl amine oxide.

(v) Cationic Surfactants

Cationic surfactants include polyethylene condensates of alkyl phenols, condensation products of ethylene oxide, propylene oxide, and ethylene oxide, propylene oxide, and ethylene diamine, long chain tertiary amine oxides, long chain tertiary phosphine oxides, and the like.

Conditioners

The conditioning compositions of this invention also contain at least a water-soluble or water-dispersible quaternary nitrogen-containing conditioning agent that is also sometimes referred to herein as a cationic compound. A long chain fatty alcohol is also present in more preferred compositions, and a tertiary amidoamine is additionally present in particularly preferred compositions.

The quaternary nitrogen-containing conditioning agents are preferably present at from about 0.5 to about 5 percent by weight of the composition as an active ingredient. More preferably, the quaternary nitrogen-containing conditioning agent is present at from about 2 to about 3 weight percent, as an active ingredient.

The class of quaternary nitrogen-containing conditioning agents useful herein contain one quaternary nitrogen atom having (a) two long aliphatic chains and (b) two identical or different short chain alkyl groups having one or two carbon atoms, each bonded to the quaternary nitrogen atom. The two long chains each contain about 12 to about 18 carbon atoms.

Illustrative conditioning agents include distearyldimethylammonium chloride and dilauryldimethylammonium chloride. These compounds are named Quaternium-5 and Quaternium-47, respectively, in the CTFA Cosmetic Ingredient Dictionary, 2nd ed., 1977, published by the Cosmetic, Toiletry and Fragrance Association, Inc., hereinafter referred to as the CTFA Dictionary.

It is noted that the long aliphatic chain of the before-mentioned conditioning agents need not be solely or primarily of one chain length, i.e., the long chain need not be cetyl, myristyl, lauryl or stearyl. Rather, conditioning agents whose long aliphatic chain contains a mixture of lengths can be used. Such conditioning agents are conveniently prepared from naturally occurring materials, such as tallow, coconut oil, soya oil and the like, or from synthetically produced mixtures. Examples of useful conditioning agents having mixed aliphatic chain lengths include dimethyldi-(hydrogenated tallow)ammonium chloride and dialkyldimethylammonium chloride wherein each alkyl group is a saturated group consisting primarily of 16 carbon atoms. These quaternary nitrogen-containing conditioning agents are named Quaternium-18 and Quaternium-31, respectively, in the CTFA Dictionary.

The compositions of this invention can also be in the form of emulsions that contain additional amounts of hydrophilic and/or hydrophobic ingredients. Emulsions containing additional hydrophobic materials are particularly preferred. It is preferred that those emulsions be stable to phase separation at a temperature of about 25. degree. C. for a period of about 24 hours after their preparation. The emulsions are more preferably stable to phase separation at temperature normally found in commercial product storage and shipping for periods of one year or more.

Water Soluble Carbonate Releasing Salt

Compositions of the invention may also contain one or more water soluble carbonate releasing salts. Nonlimiting examples of such salts include Na₂CO₃, NaHCO₃, K₂CO₃, KHCO₃, ZnCO₃, MgCO₃, Li₂CO₃, CaCO₃, (NH₄)₂CO₃, (NH₄)HCO₃, NH₄CO₂NH₂, and Ca(HCO₃)₂. These compounds maybe used singly or, as required, in combination. (NH₄)₂CO₃, (NH₄)HCO₃, NH₄CO₂NH₂, are water soluble ammonium carbonate or carbamate salts.

Water Soluble Ammonium Salt

Compositions of the invention may also contain one or more water soluble ammonium salts. Nonlimiting examples of such salts include ammonium nitrate, ammonium chloride, ammonium sulfate, ammonium bromide, ammonium phosphate, ammonium hydrogen phosphate, and mixtures thereof.

Thickeners

Thickeners may be included in compositions of the invention which have a shampoo base, and compositions of the invention which have a conditioner base, and thickeners may be included in part A and part B compositions of the invention. Long chain fatty alcohols having from about 11 to about 18 carbon atoms in the long fatty chain can be thickener constituents of the conditioning emulsions of this invention. These alcohols can be used alone, or in admixture with each other. When included in the compositions, the alcohol is preferably present at from about 0.5 to about 10 weight percent of the composition, and more preferably at from about 2 to about 5 weight percent.

Lauryl alcohol, oleyl alcohol, myristyl alcohol, stearyl alcohol, and the like, and mixtures thereof are contemplated herein. In addition, mixtures of natural or synthetic fatty alcohols having fatty chain lengths of from about 11 to about 18 carbons are also useful. Several such mixtures are available commercially, and are exemplified by the material containing a mixture of synthetic alcohols with 12 to 15 carbons in the alkyl chain sold under the trademark NEODOL 25 by Shell Chemical Company, and the material containing a mixture of synthetic alcohols with chain lengths of 12 to 16 carbons sold under the trademark ALFOL 1216 Alcohol by Conoco Chemicals.

Thickening agents suitable for use in the compositions herein may also be selected from oleic acid, cetyl alcohol, oleyl alcohol, sodium chloride, cetearyl alcohol, stearyl alcohol, synthetic thickeners such as Carbopol, Aculyn and Acrosyl and mixtures thereof. Preferred thickeners for use herein are Aculyn 22 (RTM), steareth-20 methacrylate copolymer; Aculyn 44 (RTM) polyurethane resin and Acusol 830 (RTM), acrylates copolymer that are available from Rohm and Haas, Philadelphia, Pa., USA. Additional thickening agents suitable for use herein include sodium alginate or gum arabic, or cellulose derivatives, such as methyl cellulose or the sodium salt of carboxymethylcellulose or acrylic polymers.

Fatty alcohols of the above discussed carbon chain lengths which are ethoxylated to contain an average of one or two moles of ethylene oxide per mole of fatty alcohol can be used in place of the fatty alcohols themselves. Examples of such useful ethoxylated fatty acids include ethylene glycol cetyl ether, polyoxyethylene (2) stearyl ether, and the like; the exemplary compounds having CTFA Dictionary names of Ceteth-1 and Steareth-2, respectively.

Optional Ingredients

The compositions of the present invention can comprise a wide range of optional ingredients. Examples of these functional classes include: anticaking agents, antioxidants, binders, biological additives, bulking agents, chelating agents, chemical additives, colorants, cosmetic astringents, cosmetic biocides, denaturants, drug astringents, emulsifiers, film formers, fragrance components, humectants, opacifying agents, plasticizers, preservatives, propellants, reducing agents, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful herein that are well known to one of ordinary skill in the art include solubilizing agents, sequestrants, and the like.

Other optional ingredients include organic acids. A non-exclusive list of examples of organic acids which can be used as the proton donating agent are adipic acid, tartaric acid, citric acid, maleic acid, malic acid, succinic acid, glycolic acid, glutaric acid, benzoic acid, malonic acid, salicylic acid, gluconic acid, polyacrylic acid, their salts, and mixtures thereof. Non-exclusive lists of examples of mineral acid for use herein are hydrochloric, phosphoric, sulfuric and mixtures thereof.

It has been found by experimentation that daily hair care products can achieve durable desired hair color. As noted above, the daily hair care product consists of two parts.

Part A: dye intermediates in a conditioner or shampoo base at alkaline pH

Part B: a peroxide such as hydrogen peroxide in a conditioner or shampoo base at acidic pH

Part A is mixed with part B and the recently made mixture is applied to hair. The reason current hair coloring products come in two packages is because the mixture of the coloring component and the oxidizing component is unstable and the two components must be kept apart until just before use. Similarly part A and part B of the present invention must be kept apart until just before use. By varying the concentration of the actives and the treatment time, the amount of color on hair may be varied while minimizing hair damage. To make the product more convenient and fool proof, part A and B may be packaged in dual dispensing systems where both parts are mixed outside of the package when dispensed and which is then applied to the wet hair as a conditioner or shampoo. Depending upon the amount of color desired, the treatment time could be varied from about 30 seconds to about 1 minute to about two minutes or longer.

Such conditioner or shampoo treatments would add color to hair gradually without damage due to lower contact time. Each subsequent treatment would add color until the desired shade is obtained. Depending upon the concentration of the actives and contact time, a desired shade may be reached in six to eight treatments. It has been found that since any one treatment does not exceed the threshold of irreversible damage, the total damage resulting from multiple treatments is lower than the damage from a single conventional treatment. Such a process gives the user control over the amount of color deposited on his or her hair, and also the option to discontinue further applications if the color delivered is not to his or her liking. He or she also has the option to switch to another color shade immediately without having to wait the six to eight weeks that is recommended for conventional treatments. With conventional hair color treatment, it is not recommended to perm and color hair simultaneously due to extensive damage. However, since this method colors the hair with minimum damage, perming can be done in the same time frame with this progressive coloring treatment.

The following examples, which were made, are shown as illustrations only and are not intended to limit the scope of the invention:

### EXAMPLE #1

Formula I

DI Water

70.00

Stearamidopropyl dimethylamine

0.50

Dicetyldimonium chloride/PG, 68%/27%

2.10

Stearyl alcohol and Ceteareth-20, 70%

1.00

Cetyl alcohol

3.60

DI water

3.00

Disodium EDTA

0.10

Dimethicone 100%

1.00

DC silicone fluid 245

1.80

Sodium metabisulfite

0.15

DI water

10.0

1,4-Aminophenol

0.6

4-Amino-2-hydroxytoluene

0.6

0.25

Ammonium Carbonate

8.0

PH adjust to =9.8 with 50% NaOH

Manufacturing Process:

1. Add deionized water to a flask and heat to about 180 to about 185F under argon, add EDTA and sodium metabisulfite follow by dyes.

2. Add Stearamidopropyl dimethylamine while stirring.

3. After all Stearamidopropyl dimethylamine has dissolved and the temperature in the flask is still at about 175F or above, add dicetyidimonium chloride/PG, dimethicone and DC silicone fluid 245.

4. Then add stearyl alcohol and cetereath-20 and then add cetyl alcohol.

5. When all the waxes are dispersed, increase agitator speed to moderate and mix at 180F to about 185F for about 10 minutes.

6. Let the batch cool to room temperature.

7. In a separate container prepare a solution of deionized water NaOH at room temp

8. Into the solution dissolve ammonium carbonate optionally with ammonium salt.

9. Add this separate mixture into the main batch at room temperature and stir for 30 minutes.

10. Adjust the pH of the resulting mixture to between about 9.7 and 10.1.

### EXAMPLE#2

Formula 2

DI Water

70.00

Stearamidopropyl dimethylamine

0.50

Dicetyidimonium chloride/PG, 68%/27%

2.10

Stearyl alcohol and Ceteareth-20, 70%

1.00

Cetyl alcohol

3.60

DI water

3.00

Disodium EDTA

0.10

Dimethicone 100%

1.00

DC silicone fluid 245

1.80

Sodium metabisulfite

0.15

DI water

10.0

p-Phenylenediamine

0.8

Resorcinol

0.8

Ammonium Carbonate

8.0

PH adjust to =9.8 with 50% NaOH

Peroxide conditioner: Part B

| | |
|---|---|
| D.I. Water | 74.00 |
| Liquid Citric acid, 50% | 0.20 |
| Stearamidopropyl dimethylamine | 0.50 |
| Dicetyldimonium chloride/PG, 68%/27% | 2.10 |
| Stearyl alcohol and Ceteareth-20, 70% | 1.00 |
| Cetyl alcohol | 3.80 |
| DI water | 5.00 |
| Disodium EDTA | 0.10 |
| Dimethicone 100% | 1.00 |
| DC silicone fluid 245 | 1.80 |
| Hydrogen Peroxide (35%) | 10.00 |
| DMDM Hydantoin 55% | 0.10 |
| Fragrance | 0.20 |
| Phosphoric acid, 85% | 0.09 |

PH=2.5 to 3.5

Manufacturing Process:

1. Add item#1 into a suitable container. Heat to 180-185F

2. Add item#2 with moderate agitation

3. Add item#4, dissolve completely

4. At 165-170F, add item#3,5,6

5. Mix batch at approximately 180-185F for at least an hour with agitation until particles are completely dissolved. Check for the particles

6. Begin cooling.

7. In a separate container dissolve item#8 in item#7)

8. At 130F add the EDTA solution to the batch.

9. At 110F or below, add item# 9, 10, 11, 12, and 13 Mix well

10. Adjust the pH with item#14 to 2.7 to 3.0

11. Qs with water

Method for Using Compositions of the Invention

Shampoo the hair as usual. Pump the composition of the invention into the palm and mix. Apply product to the hair like a regular conditioner. Rinse the hands. Leave product in the hair for example for about 30 seconds to about 2 minutes for color maintenance; and for example for about 5 minutes for color change. Rinse the hair thoroughly.

Experimental

1. Formulation Stability: It is generally accepted that a marketed formulation for Hair Color needs to be stable for at least 3 months at 45C to survive market conditions globally. The formulations tested using carbonate salts as high as 10% in combination with CTAB can be shown to meet these criteria for stability.

2. Hair Tress Lightening (Bleaching): Caucasian hair tresses either Dark or medium brown were supplied from IHS. These tresses were about 9 inches in length and weigh about 9 grams. Hair Color measurements (L,a,b) were taken before and after treatment with a formulation using a Hunter Labscan XE. Each tress was first wet with water and treated with a formulation not containing dyes in a ratio of 1:1 for as short as 2 minutes and as long as 45 minutes. At least 3 tresses were used per formulation. Multiple treatments were also conducted to observe changes in lightening over time. These were generally on the order of 10, 20 and 30 consecutive treatments. All tresses were rinsed in water to remove any residual product and then measured for changes in lightening.

The results using a statistical design indicated that a Hair Coloring formula containing ammonium carbonate as low as 2% at pH 9.5 could be shown to lighten hair significantly better than a similar formula containing an equal molar weight of ammonia at pH 9.5. This was demonstrated for tresses treated singly for 45 minutes or after 10, 20 or 30 applications for 2 minutes each.

3. Trained Panel Assessment: Nine panelists trained in assessing damage of hair tresses were blinded in their evaluation of tresses treated with either ammonium carbonate or an ammonia containing Hair Coloring formulation. Tresses were treated 30 times for 2 minutes each with either of these formulations. The results indicated that the trained panelists ranked the ammonium carbonate tresses to be significantly less damaged than a similar formulation containing ammonia.

4. Color Enhancement: When Hair Coloring formulations contained oxidative dyes as PAP and PAOC, it could be shown that piedmont hair treated with a formulation containing ammonium carbonate deposited more color than a similar formulation with ammonia as measured visually or using a Color instrument as the Hunter.

5. Mechanical Measurement of Tensile Properties of Hair Treated With Rainbow Formula:

These studies employed standard instrument from Diastron—automated tensile tester model MTT 670. Prior to testing fiber diameters were assessed using commercially available Mitutoyo Laser Scan Micrometer. Single hair fiber tests were carried out on hair soaked in water for 30 min. During test hair fiber (gauge length of 30 mm) was stretched at an extension rate of 20 mm/min during which load versus extension curves were recorded. The average of 60 hairs was used for each data point and four parameters of the tensile curves were compared:

elastic modulus (slope of the linear portion of the curve, N/m²)

break extension (extent of hair stretching before break, % of hair length extension)

break stress (force needed to break fiber, N/m²)

work to 20% extension (MJ/m²)

The results indicated that the changes in all tensile strength parameters on hair treated with a formulation containing ammonium carbonate are similar to the changes induced to the fiber by similar formulation containing ammonia. The carbonate treated hair can be shown being not more damaging than the formulation containing ammonia.

6. Combing force measurements were performed on an Instron 5542 tensile tester. A metal block which has a groove cut where the comb is placed, was secured to the base of tester with screws and was used as the comb holder in these combing force measurements. Each tress was manually combed three times (so that no tangles are left) before measurement. Root portion of hair tress was then mounted in a rigid clip secured to the movable cross-head of tester and free hanging portion of hair was pressed through to the base of the comb teeth. Position of the tress during combing was secured by the metal deflector inserted in front of the tress. 3.5 g tresses of 5 in length were used for evaluation. 2.5 g of product per 1 g hair was applied on hair for 5 min. After rinsing, dry and wet tress weight were compared and excess water squeezed out to minimize the variation in moisture content between tresses. Immediately afterwards measurement on wet tresses was performed. Dry combing is performed on tresses after a free-hanging drying for 24 hrs. Combing work (kgf) for each tress was calculated from the force (kg-mm) needed to comb through the tress over its entire length. Tests included one internal control—untreated hair tress wetted under the condition used for product application. From the results of these tests it could be seen that compositions of the invention which comprised ammonium carbonate had similar conditioning properties as similar compositions which did not have ammonium carbonate.

At the same time that hair damage is avoided by the methods of the invention, the consumer's hair is gradually being brought to the desired shade and color. This gradual change of color has three advantages: first, since the color is changed gradually, the consumer can stop the process if he or she does not like the color his or her hair is turning to. Second, some consumers do not want an abrupt change in color because they may feel embarrassed in public after having made such an abrupt change to the color of their hair. The methods of the present invention avoid such abrupt hair color changes. Third, the methods and compositions of the present invention can be used in the shower, and on a daily basis, because the compositions and methods of the present invention, by contrast with current color compositions, do not employ poisonous levels of chemicals and also because compositions and methods of the present invention involve hair application steps that are usually up to about two minutes to five minutes in length. By contrast current hair coloring compositions require approximately 30 minutes' time for each application, an amount of time, which is clearly not suitable for use in the shower.

The methods of the present invention are not as messy as conventional permanent hair coloring methods. The methods of the present invention do not use chemical compositions that are as smelly and noxious as those used in conventional permanent hair-coloring methods. Because the methods of the invention can be carried out in the shower, they do not involve the dripping and the mess associated with conventional permanent hair coloring. The methods of the present invention do not use chemical compositions that can stain fixtures in the bathroom or that will stain the scalp and the face and the hands. Parts A and B upon mixture form a composition with a pleasing viscosity and that is pleasing to the fingers. This is because one or both of Parts A and B contain a cationic conditioning agent.

A dual package which can be employed in the products and kits of the present invention is disclosed in U.S. Pat. No. 6,082,588 to Markey et al which is hereby incorporated by reference.

Kit Containing an Instruction Sheet

The present invention also relates to a kit for carrying out the hair coloring method of the invention. The kit comprises a developer solution, a dye solution and a post treatment solution, each in a separate container or in a dual container, as described herein. The kit also contains written instructions that explain how the compositions of the invention are used.

The consumer admixes the components of the kit according to written instructions, to obtain the aqueous reaction mixture. The admixture may be conducted in a separate vessel external to the kit, or may take place in a container of the kit adapted to provide sufficient head space for mixing. The components that are mixed are the developer composition part B; and the dye composition, part A. The reactants may also be admixed on the hair of the user. Essentially upon mixing, reaction of the dye and the developer will commence. After treatment for a desired time the mixture of hair developer part B and hair dye part A is removed, preferably with water or a conventional shampoo or a conventional conditioning shampoo.

As noted above, dye intermediate composition and peroxygen composition are mixed together, and the resulting mixture is applied to the hair and allowed to remain for a set time, usually about 30 seconds to about 1 to about 2 minutes to about 5 minutes or more.

Measurement of the change in hair color may be achieved in a number of other ways. In the first instance, the consumer can compare his or her hair color with desired hair color or the hair color of a sample tress. Hair dyeing by the method of the invention can be repeated until her hair color matches the desired hair color.

By the method of the invention, durable desired, effective hair color, with longer lasting hair condition, clean soft feel, and a minimum of hair damage is achieved.

In following the method of the invention, the consumer can compare the color of her hair with the desired hair color, which can be printed on the package of the product. The consumer can also vary the number of days of application of the product, and the consumer can also vary the amount of time the mixture of composition 1 and 2 is left in the hair on each application. The number of applications can vary from about 7 to about 30 applications. The time of each application can vary from about 1 to about two minutes.

The method of the invention occurs over the course of days. Therefore, the final color of the consumer's hair may be affected by the amount of exposure to the sun of the hair during the course of treatment.

Desired hair color can also be reached by comparing hair after each treatment until it matches hair tresses taken from the consumer during a prior treatment.

Desired hair color can also be reached by testing the hair after each treatment with instruments, which measure the color of the hair. When the measurements of hair color of the treated hair reach a desired level, the treatment hair reach a desired level, the treatment can be stopped.

Indeed, reaching the desired hair color can be achieved by the use of any matching or comparison method commonly employed in the art.

It is noted that the compositions used in the methods of the invention have lower contact times and thus repeated use of these compositions will not cause hair damage. An unexpected discovery of the present invention is that for damage to hair to occur, contact time in each treatment must go above the threshold value of about two minutes. Thus, the consumer can lighten or color the hair through repeated applications wherein the duration of each color application is about two minutes or less. The following two advantages are thereby achieved: first, there is a stepwise approach to the desired color; and second, a minimum of hair damage is done.

What is claimed is:

1. A method for permanently dyeing hair which comprises subjecting said hair to a number of treatments, having a set time interval between each two consecutive such treatments, wherein each treatment comprises steps a.) and b.) below:
a.) contacting said hair, for a period of about 5 seconds to about 5 minutes with a
recently made mixture of:
I an alkaline composition comprising a dye intermediate in a shampoo base or in a conditioner base and
II
p) a water soluble carbonate releasing salt at about 0.1% to about 15%, more preferably about 1% to about 10%, and more preferably about 4% to about 10%, and most preferably about 8%; and
vii) a water soluble ammonium salt at about 0.1% to about 15%, more preferably about 1% to about 10%, and more preferably about 4% to about 10%, and most preferably about 8%; or
II
a water soluble ammonium carbonate or carbamate salt at about 0.1% to about 15%, more preferably about 1% to about 10%, and more preferably about 4% to about 10%, and most preferably about 8%; and
III
Part B: A peroxide compound such as hydrogen peroxide in a conditioner or shampoo base at acidic pH;
b.) rinsing said mixture from said hair with water;
with the proviso that when a conditioner base is present in a.) i.) above, an independently selected conditioner base is also present in a.) ii.) above; and when a shampoo base is present in a.) i.) above, an independently selected shampoo base is also present in a.) ii.) above;
b.) rinsing said mixture from said hair with water;
with the proviso that when a conditioner base is present in a.) i.) above, an independently selected conditioner base is also present in a.) ii.) above; and when a shampoo base is present in a.) i.) above, an independently selected shampoo base is also present in a.) ii.) above;
and wherein said number of treatments is between about 2 to about 30; and wherein said set time interval between each two consecutive treatments is between about 8 hours and 30 days.

2. A method according to claim 1 wherein Part A comprises from
a) about 0.1 to about 99.9% of a conditioning base, wherein the conditioning agent within said conditioning base comprises from about 0.5 to about 10% of the total composition % of a conditioner;
b) about 0.1 to about 5% of oxidation dyestuffs; and
c) about 0.1 to about 5% of a coupling compound

3. A method according to claim 1, wherein said dye intermediate is selected from the group consisting of m-aminophenol, p-phenylene diamine, :p-toluenediamine; p-phenylenediamine; 2-chloro-p-phenylenediamine; N-phenyl-p-phenylenediamine; N-2-methoxyethyl-p-phenylenediamine; N,N-bis-(hydroxyethyl)-p-phenylenediamine; 2-hydroxymethyl-p-phenylenediamine; 2-hydroxyethyl-p-phenylenediamine; 4,4′-diaminodiphenylamine; 2,6-dimethyl-p-phenylenediamine; 2-isopropyl-p-phenylenediamine; N-(2-hydroxypropyl)-p-phenylenediamine; 2-propyl-p-phenylenediamine; 1,3-N,N-bis-(2-hydroxyethyl)-N,N-bis (4-aminophenyl)-2-propanol; 2-methyl-4-dimethylaminoaniline; p-aminophenol; p-methylaminophenol; 3-methyl-p-aminophenol; 2-hydroxymethyl-p-aminophenol; 2-methyl-p-aminophenol; 2-(2-hydroxyethylaminomethyl)-p-aminophenol; 2-methoxymethyl-p-aminophenol; and 5-aminosalicylic acid; catechol; pyrogallol; o-aminophenol; 2,4-diaminophenol; 2,4,5-trihydroxytoluene; 1,2,4-trihydroxybenzene; 2-ethylamino-p-cresol; 2,3-dihydroxynaphthalene; 5-methyl-o-aminophenol; 6-methyl-o-aminophenol; and 2-amino-5-acetaminophenol; 2-methyl-1-naphthol; 1-acetoxy-2-methylnaphthalene; 1,7-dihydroxynaphthalene; resorcinol; 4-chlororesorcinol; 1-naphthol; 1,5-dihydroxynaphthalene; 2,7-dihydroxynaphthalene; 2-methylresorcinol; 1-hydroxy-6-aminonaphthalene-3-sulfonic acid; thymol (2 isopropyl-5-methylphenol); 1,5-dihydroxy-1,2, 3,4-tetrahydronaphthalene; 2-chlororesorcinol; 2,3-dihydroxy-1,4-naphthoquinone; and 1-naphthol-4-sulfonic acid; m-phenylenediamine; 2-(2,4-diaminophenoxy)ethanol; N,N-bis(hydroxyethyl)-m-phenylenediamine; 2,6-diaminotoluene; N,N-bis(hydroxyethyl)-2,4-diaminophenetole; bis(2,4-diaminophenoxy)-1,3-propane; 1-hydroxyethyl-2,4-diaminobenzene; 2-amino-4 hydroxyethylaminoanisole; aminoethoxy-2,4-diaminobenzene; 2,4-diaminophenoxyacetic acid; 4,6-bis(hydroxyethoxy)-m-phenylenediamine; 2,4-diamino-5-methylphenetole; 2,4-diamino-5-hydroxyethoxytoluene; 2,4-dimethoxy 1,3-diaminobenzene; 2,6-bis(hydroxyethylamino) toluene; m-aminophenol; 2-hydroxy-4-carbamoylmethylaminotoluene; m-carbamoylmethylaminophenol; 6-hydroxybenzomorpholine; 2-hydroxy-4-aminotoluene; 2-hydroxy-4-hydroxyethylaminotoluene; 4,6-dichloro-maminophenol; 2-methyl-m-aminophenol; 2-chloro-6-methyl-m-aminophenol; 2-hydroxyethoxy-5-aminophenol; 2-chloro-5-trifluoroethylaminophenol; 4-chloro-6-methyl-m-aminophenol; N-cyclopentyl-3-aminophenol; N-hydroxyethyl-4-methoxy-2-methyl-m-aminophenol and 5-amino-4-methoxy-2-methylpheno; 2-dimethylamino-5-aminopyridine; 2,4,5,6-tetraminopyrimidine; 4,5-diamino-1-methylpyrazole; 1-phenyl-3-methyl-5-pyrazolone; 6-methoxy-8-aminoquinoline; 2,6-dihydroxy-4-methylpyridine; 5-hydroxy-1,4-benzodioxane; 3,4-methylenedioxyphenol; 4-hydroxyethylamino-1,2-methylenedioxybenzene; 2,6-dihydroxy-3,4-dimethylpyridine; 5-chloro-2,3-dihydroxypyridine; 3,5-diamino-2,6-dimethoxypyridine; 2-hydroxyethylami no-6-methoxy-3-aminopyridine; 3,4-methylenedioxyaniline; 2,6-bis-hydroxyethoxy-3,5-diaminopyridine; 4-hydroxyindole; 3-amino-5-hydroxy-2,6-dimethoxypyridine; 5,6-dihydroxyindole; 7-hydroxyindole; 5-hydroxyindole; 2-bromo-4,5-methylenedioxyphenol; 6-hydroxyindole; 3-amino-2-methylamino-6-methoxypyridine; 2-amino-3-hydroxypyridine; 2,6-diaminopyridine; 5-(3,5-diamino-2-pyridyloxy)-1,3-dihydroxypentane; 3-(3,5-diamino-2-pyridyloxy)-2-hydroxypropanol and 4-hydroxy-2,5,6-triaminopyrimidine, or combinations thereof.

4. A method according to claim 1, wherein said conditioner base in a.) i.) comprises a conditioning agent independently selected from the group consisting of dicetyl dimonium chloride/PG selected from the group consisting of poly(dimethyldiallylammonium chloride); Quaternium 57; poly(dipropyldiallylammonium chloride), poly(methyl-beta-propaniodiallylammonium choride), poly(diallylpiperidinium chloride), ly(vinylpyridinium chloride), quaternised poly(vinyl alcohol) and quaternised poly (dimethylaminoethylmethacrylate), as poly (N-vinylpyrollidone), poly(dimethylaminoethylmethacrylate), poly(vinyl pyridine) and poly(ethyleneimine) and mixtures thereof; and wherein said conditioner base in a.) ii.) comprises a conditioning agent independently selected from the group consisting of dicetyl dimonium chloride/PG selected from the group consisting of poly(dimethyldiallylammonium chloride); Quaternium 57; poly(dipropyldiallylammonium chloride), poly(methyl-beta-propaniodiallylammonium choride), poly(diallylpiperidinium chloride), ly(vinylpyridinium chloride), quaternised poly(vinyl alcohol) and quaternised poly (dimethylaminoethylmethacrylate), as poly (N-vinylpyrollidone), poly(dimethylaminoethylmethacrylate), poly(vinyl pyridine) and poly(ethyleneimine), and mixtures thereof.

5. A method according to claim 1, wherein said alkaline composition of a.) i.) prior to mixture with said acidic mixture of a.)ii.) has a pH of about 3 to about 5.

6. A method according to claim 1, wherein said acidic composition of a.) ii.) prior to mixture with said alkaline mixture of a.) i.) has a pH of about 8 to about 10.

7. A method according to claim 1 wherein said alkaline composition according to a.) i.) comprises:
A.) from about 0.05% to about 1.0% of a dye intermediate;
B.) from about 0.1% to about 0.5% of a coupler; and
C.) from about 1% to about 90% of a conditioner base.

8. A method according to claim 1 wherein said acidic composition according to a.) ii.) comprises:
A.) from about 1% to about 90% of a conditioner base;
B.) from about 0.5% to about 2.5% of a volatile silicone; and
C.) from about 0.1% to about 5% of an oxidative compound.

9. A method according to claim 1 wherein said period for contacting said hair is between about 1 minute and 3 minutes.

10. A method according to claim 1 wherein said set time interval is between about 1 day and about 3 days.

11. A method according to claim 1 wherein said hair is highlighted.

12. A method according to claim 1 wherein said hair has wet combing tensile strength maintained.

13. A method according to claim 1 wherein said hair is not significantly damaged.

14. A method according to claim 1 wherein said hair is conditioned.

15. A method according to claim 1 wherein said hair is shampooed.

16. A method according to claim 1 wherein said oxidative compound is selected from the group consisting of hydrogen peroxide, urea peroxide, melamine peroxide, sodium perborate and sodium percarbonate.

17. A method according to claim 1 wherein the first composition comprises from about 35% to about 98.9% water.

18. A method according to claim 1, wherein the mixture of part A and part B has a neat viscosity of from about 500 cps to about 60,000 cps at 26.7.degree. C., as measured by a Brookfield RVTDCP with a spindle CP-41 at 1 RPM for 3 minutes.

19. A method for maintaining hair color through the use of a permanent hair dye which comprises subjecting said hair to successive treatments, having a set time interval between each two consecutive such treatments, wherein each treatment comprises steps a.) and b.) below:
a.) contacting said hair, for a period of about 5 seconds to about 5 minutes with a
recently made mixture mixture of:
I an alkaline composition comprising a dye intermediate in a shampoo base or in a conditioner base and
II
q) a water soluble carbonate releasing salt at about 0.1% to about 15%, more preferably about 1% to about 10%, and more preferably about 4% to about 10%, and most preferably about 8%; and
viii) a water soluble ammonium salt at about 0.1% to about 15%, more preferably about 1% to about 10%, and more preferably about 4% to about 10%, and most preferably about 8%; or
II
a water soluble ammonium carbonate or carbamate salt at about 0.1% to about 15%, more preferably about 1% to about 10%, and more preferably about 4% to about 10%, and most preferably about 8%; and
III
Part B: A peroxide compound such as hydrogen peroxide in a conditioner or shampoo base at acidic pH;
b.) rinsing said mixture from said hair with water;
ii.) with the proviso that when a conditioner base is present in a.) i.) above, an independently selected conditioner base is also present in a.) ii.) above; and when a shampoo base is present in a.) i.) above, an independently selected shampoo base is also present in a.) ii.) above;
b.) rinsing said mixture from said hair with water;
with the proviso that when a conditioner base is present in a.) i.) above, an independently selected conditioner base is also present in a.) ii.) above; and when a shampoo base is present in a.) i.) above, an independently selected shampoo base is also present in a.) ii.) above;
and wherein said number of treatments is at least about 2; and wherein said set time interval between each two consecutive treatments is between about 8 hours and 30 days.

20. A method according to claim 1 wherein said dye intermediate is present at about 0.1% to about 1%.

21. A method according to claim 1 wherein said oxidative compound is present at about 2% to about 5%.

22. A dispenser for dispensing simultaneously composition a.) i.) and composition a.) ii.) according to claim 1, which comprises:
A.) a means for holding composition a.) i.) and composition a.) ii.) in physically separate locations;
B.) a means for protecting composition a.) i.) and composition a.) ii.) from air prior to dispensing;
C.) a means for dispensing composition a.) i.) and composition a.) ii.) in equal amounts and in proximity to each other.

23. A method according to claim 1 wherein composition a.) i.) and composition a.) ii.) are mixed by hand.

24. A method according to claim 1 which comprises rinsing said mixture of composition a.) i.) and composition a.) ii.) from said hair with water in a shower.

25. A composition for permanently dying hair which comprises a mixture of:
I an alkaline composition comprising a dye intermediate in a shampoo base or in a conditioner base and
II
r) a water soluble carbonate releasing salt at about 0.1% to about 15%, more preferably about 1% to about 10%, and more preferably about 4% to about 10%, and most preferably about 8%; and
ix) a water soluble ammonium salt at about 0.1% to about 15%, more preferably about 1% to about 10%, and more preferably about 4% to about 10%, and most preferably about 8%; or
II
a water soluble ammonium carbonate or carbamate salt at about 0.1% to about 15%, more preferably about 1% to about 10%, and more preferably about 4% to about 10%, and most preferably about 8%; and
III
Part B: A peroxide compound such as hydrogen peroxide in a conditioner or shampoo base at acidic pH;

26. A composition according to claim 25 which further comprises in part A, part B, or part A and part B, a thickener which is a high molecular weight fatty alcohol.

27. A composition according to claim 26 wherein said high molecular weight fatty alcohol is selected from the group consisting of cetyl alcohol and stearyl alcohol.

28. A composition for permanently dying hair which comprises a mixture of
Part A
a) about 0.1% to about 99.9% of a shampoo base; and
b) about 0.1% to about 1% of a dye;
Part B
a) about 1 to about 5% of a shampoo base;
b) about 1 to about 5% of an oxidative compound.

29. A composition according to claim 28 wherein said shampoo base in part A comprises about 5 to about 50%, based on the total part A composition, of an anionic surfactant, an amphoteric surfactant, or a mixture of an anionic surfactant and an amphoteric surfactant.

30 A composition according to claim 25, which has a pH of about 9 to about 11.

31 A composition according to claim 25 wherein the water soluble carbonate releasing salt is selected from the group consisting of Na₂CO₃, NaHCO₃, K₂CO₃, KHCO₃, ZnCO₃, MgCO₃, Li₂CO₃, CaCO₃, (NH₄)₂CO₃, (NH₄)HCO₃, NH₄CO₂NH₂, and Ca(HCO₃)₂ and mixtures thereof.

32 A composition according to claim 25 wherein the water soluble ammonium salt is selected from the group consisting of ammonium chloride, ammonium carbonate, ammonium bicarbonate, ammonium sulfate, ammonium hydrogen phosphate, and mixtures thereof.

33 A composition according to claim 25 which comprises about 0.1 to about 12% an carbonate salt, or about 0.1 to about 12% of a metal carbonate salt and about 0.1 to about 12% of a non-carbonate ammonium salt.

34 A composition according to claim 25 which comprises about 1 to about 10% an carbonate salt, or about 1 to about 10%, of a metal carbonate salt and about 1 to about 10%, ammonium salt.

35 A composition according to claim 25 which comprises about 4 to about 10% an carbonate salt, or about 4 to about 10%, of a metal carbonate salt and about 4 to about 10%, ammonium salt.

36 A composition according to claim 28 which comprises:

| Ingredients | % w/w |
|---|---|
| Oxidative dyes | 0.01-15 |
| Sequestrant | 0.01-1 |
| Antioxidant | 0.01-3 |
| Solvent | 2.0-35 |
| Buffering agent | 0.01-10 |
| Thickener | 0.05-20 |
| Surfactants | 5.0-40 |
| Water soluble carbonate releasing salt | 1.0-15 |
| Water soluble ammonium salt | 0-15 |
| Water to | 100 |
